(19) United States
(12) Patent Application Publication
(10) Pub. No.: US 2010/0160749 A1
(43) Pub. Date: Jun. 24, 2010

(54) IMPLANTABLE OPTICAL GLUCOSE SENSING

(75) Inventors: Yossi GROSS, Moshav Mazor (IL); Tehila Hyman, Modi'in (IL); Tamir Gil, Meuchad (IL)

Correspondence Address: BIRCH STEWART KOLASCH & BIRCH, PO BOX 747, FALLS CHURCH, VA, 22040-0747, US

(73) Assignee: GLUSENSE LTD., Lod (IL)

(21) Appl. No.: 12/344,103

(22) Filed: Dec. 24, 2008

(51) Int. Cl. A61B 5/1459

(52) U.S. Cl. 600/316

(57) ABSTRACT

Apparatus is provided, including a support configured to be implanted within a body of a subject and a sampling region coupled to the support. The apparatus is configured to passively allow passage through the sampling region of at least a portion of fluid from the subject. The apparatus also comprises an optical measuring device in optical communication with the sampling region. The optical measuring device comprises at least one light source configured to transmit light through at least a portion of the fluid, and at least one sensor configured to measure a parameter of the fluid by detecting light passing through the fluid. Other embodiments are also described.

### CROSS-REFERENCES TO RELATED APPLICATIONS

The present application is related to:

U.S. Provisional Patent Application 60/588,211 to Gross et al., entitled, “Implantable sensor,” filed Jul. 14, 2004;

U.S. Provisional Patent Application 60/658,716 to Gross et al., entitled, “Implantable fuel cell,” filed Mar. 3, 2005;

PCT Patent Application PCT/IL2005/000743 to Gross et al., entitled “Implantable power sources and sensors,” filed Jul. 13, 2005;

U.S. Provisional Patent Application 60/786,532 to Gross et al., entitled, “Implantable sensor,” filed Mar. 28, 2006;

PCT Patent Application PCT/IL2007/000399 to Gross, entitled “Implantable sensor,” filed Mar. 28, 2007.

All of these applications are incorporated herein by reference.

### FIELD OF THE INVENTION

The present invention relates generally to implantable sensors and specifically to methods and apparatus for sensing blood glucose concentrations.

### BACKGROUND OF THE INVENTION

Diabetes mellitus is a disease in which cells fail to uptake glucose either due to a lack of insulin (Type I) or an insensitivity to insulin (Type II). The associated elevation of blood glucose levels for prolonged periods of time has been linked to a number of problems including retinopathy, nephropathy, neuropathy, and heart disease. A typical care regimen for Type I diabetics includes daily monitoring of blood glucose levels and injection of an appropriate dose of insulin. Conventional glucose monitoring involves the use of an invasive “finger-stick” method in which the finger of a subject is pricked in order to withdraw a small amount of blood for testing in a diabetes monitoring kit based on the electro enzymatic oxidation of glucose.

Polarimetric measurement of glucose concentration is based on optical rotatory dispersion (ORD) a phenomenon by which a solution containing a chiral molecule rotates the plane of polarization for linearly polarized light passing through it. The rotation is the result of a difference in refractive indices nL and nR for left and right circularly polarized light traveling through the electron cloud of a molecule.

US Patent Application Publication 2007-0066877 to Arnold et al., which is incorporated herein by reference, describes an implantable microspectrometer for the reagentless optical detection of an analyte in a sample fluid. The microspectrometer comprises an optical sampling cell having a cell housing defining a fluid inlet port and a fluid outlet port, the fluid inlet port configured to receive an optical sampling fluid from a test subject; an electromagnetic radiation source in communication with a first portion of the optical sampling cell housing and configured to irradiate at least a portion of the optical sampling fluid with electromagnetic radiation; and an electromagnetic radiation detector in communication with a second portion of the optical sampling cell housing and configured to detect electromagnetic radiation emanating from the optical sampling cell. In use, the implantable microspectrometer can optically detect at least one parameter of an analyte contained within the optical sampling fluid in the absence of an added reagent.

U.S. Pat. No. 6,049,727 to Crothall, describes an in vivo implantable sensor which obtains spectra of body fluid constituents and processes the spectra to determine the concentration of a constituent of the body fluid. The sensor includes an optical source and detector. The source emits light at a plurality of different, discrete wavelengths, including at least one wavelength in the infrared region. The light interacts with the body fluid and is received at the detector. The light at the plurality of different wavelengths has a substantially collinear optical path through the fluid with respect to each other. When measuring fluid constituents in a blood vessel, such as blood glucose, the light at the plurality of different wavelengths is emitted in a substantially single period of time. The spectra are corrected for artifacts introduced from extraneous tissue in the optical path between the source and the detector. The sensor is fully implanted and is set in place to allow plural measurements to be taken at different time periods from a single in vivo position. The light source emits at least three different wavelengths.

U.S. Pat. No. 6,577,393 to Potzschke et al., describes a process or device for determination of the plane of polarization of polarized light. Light from a light source is polarized by means of a polarizing filter which has a certain setting angle .theta..sub.0 with respect to the first reference plane, the plane of incidence on the reflecting surface. The polarized beam passes through the sample in the measurement chamber, in which the angle of rotation is changed by the small angle .theta..sub.MG, The sum of .theta..sub.0 and .theta..sub.MG gives the angle of rotation .theta..sub.e, at which the beam emerging from the measurement chamber is partially reflected at the surface of a medium of higher refractive index. The reflected beam is then separated into two partial beams (an extraordinary beam and an ordinary beam), with vibration directions exactly perpendicular to each other. In a polarizing prism, the reference plane of which, the plane of vibration of the ordinary beam, has a certain setting angle (.theta.*) with respect to the first reference plane. The intensities I.sub.o and I.sub.a of the two partial beams are determined photometrically by detectors and the ratio of the measured intensities is determined by a quotient determiner.

U.S. Pat. No. 5,209,231 to Cote et al., describes optically based apparatus for non-invasively determining the concentration of optically active substances in a specimen. The apparatus comprises a source of a beam of spatially coherent light which is acted upon to produce a rotating linear polarized vector therein. A beam splitter splits the beam into a reference beam and a detector beam for passage through the specimen. The detector beam is received upon exiting the specimen and compared with the reference beam to determine the amount of phase shift produced by passage through the specimen. This amount of phase shift is converted into concentration of the optically active substance in the specimen.

U.S. Pat. No. 6,188,477 to Pu et al., describes integrated polarization sensing apparatus and method uses a self-homodyne detection scheme to provide required sensitivity for numerous applications, such as glucose concentration monitoring, without the need for expensive, bulky components. The detection scheme is implemented by splitting a polarized laser beam with a polarization beam splitter into a P wave component and an S wave component, phase modulating the P wave component and recombining the two components. The polarization of the combined optical beam is then rotated slightly by the variable to be monitored, such as by passing the beam through a glucose solution. Finally, the beam is passed onto a photodetector that generates a signal that is proportional to the polarization rotation angle. This device has the advantage of employing optical components, including polarizing beam splitters, phase modulators and lenses, that can all be fabricated on a single silicon chip using MEMS technology so that the device can be made compact and inexpensive.

U.S. Pat. No. 6,061,582 to Small et al., describes non-invasive measurements of physiological chemicals such as glucose in a test subject using infrared radiation and a signal processing system. The level of a selected physiological chemical in the test subject is determined in a non-invasive and quantitative manner by a method comprising the steps of: (a) irradiating a portion of the test subject with near infrared radiation; (b) collecting data concerning the irradiated light on the test subject; (c) digitally filtering the collected data to isolate a portion of the data indicative of the physiological chemical; and (d) determining the amount of physiological chemical in the test subject by applying a defined mathematical model to the digitally filtered data. The collected data is in the form of either an absorbance spectrum or an interferogram.

U.S. Pat. No. 6,587,704 to Fine et al., describes a non-invasive method of optical measurements for determining at least one desired parameter of patient's blood. The method utilizes reference data indicative of the values of the desired blood parameter as a function of at least two measurable parameters. At least one of the measurable parameters is derived from scattering spectral features of the medium highly sensitive to patient individuality, and the at least one other measurable parameter is indicative of artificial kinetics of the optical characteristics of the patient's blood perfused fleshy medium. A condition of artificial kinetics is created at a measurement location, and maintained during a certain time. Measurements are carried out with different wavelengths of incident light during a time period including this certain time. Measured data is in the form of time evolutions of light responses of the medium corresponding to the different wavelengths. By analyzing the measured data, the at least two measurable parameters are extracted, and the reference data is utilized to determine the at least one desired blood parameter.

US Patent Application Publication 2007-0004974 to Nagar et al., describes apparatus for assaying an analyte in a body comprising: at least one light source implanted in the body controllable to illuminate a tissue region in the body with light at at least one wavelength that is absorbed by the analyte and as a result generates photoacoustic waves in the tissue region; at least one acoustic sensing transducer coupled to the body that receives acoustic energy from the photoacoustic waves and generates signals responsive thereto; and a processor that receives the signals and processes them to determine a concentration of the analyte in the illuminated tissue region.

U.S. Pat. No. 3,837,339 to Aisenberg et al., describes techniques for monitoring blood glucose levels, including an implantable glucose diffusion-limited fuel cell. The output current of the fuel cell is proportional to the glucose concentration of the body fluid electrolyte and is therefore directly indicative of the blood glucose level. This information is telemetered to an external receiver which generates an alarm signal whenever the fuel cell output current exceeds or falls below a predetermined current magnitude which represents a normal blood glucose level. Valve means are actuated in response to the telemetered information to supply glucose or insulin to the monitored living body.

U.S. Pat. Nos. 5,368,028 and 5,101,814 to Palti, describe methods and apparatus for monitoring blood glucose levels by implanting glucose sensitive living cells, which are enclosed in a membrane permeable to glucose but impermeable to immune system cells, inside a patient. Cells that produce detectable electrical activity in response to changes in blood glucose levels are used in the apparatus along with sensors for detecting the electrical signals, as a means for detecting blood glucose levels. Human beta cells from the islets of Langerhans of the pancreas, sensor cells in taste buds, and alpha cells from the pancreas are discussed as appropriate glucose sensitive cells.

Methods for immunoprotection of biological materials by encapsulation are described, for example, in U.S. Pat. Nos. 4,352,883, 5,427,935, 5,879,709, 5,902,745, and 5,912,005. The encapsulating material is typically selected so as to be biocompatible and to allow diffusion of small molecules between the cells of the environment while shielding the cells from immunoglobulins and cells of the immune system. Encapsulated beta cells, for example, can be injected into a vein (in which case they will eventually become lodged in the liver) or embedded under the skin, in the abdominal cavity, or in other locations. Fibrotic overgrowth around the implanted cells, however, gradually impairs substance exchange between the cells and their environment. Hypoxia of the cells typically leads to cell death.

PCT Patent Publication WO 2006/006166 to Gross et al., which is incorporated herein by reference, describes a protein, including a glucose binding site, cyan fluorescent protein (CFP), and yellow fluorescent protein (YFP). The protein is described as being configured such that binding of glucose to the glucose binding site causes a reduction in a distance between the CFP and the YFP. Apparatus is also described for detecting a concentration of a substance in a subject, the apparatus comprising a housing adapted to be implanted in the subject. The housing comprises an optical detector, and cells genetically engineered to produce, in a patient's body, a FRET protein comprising a fluorescent protein donor, a fluorescent protein acceptor, and a protein containing a binding site for the substance.

United States Patent Application Publication 2005/0118726 to Schultz et al., describes a method for making a fusion protein, having a first binding moiety having a binding domain specific for a class of analytes that undergoes a reproducible allosteric change in conformation when said analytes are reversibly bound; a second moiety and third moiety that are covalently linked to either side of the first binding moiety such that the second and third moieties undergo a change in relative position when an analyte of interest molecule binds to the binding moiety; and the second and third moieties undergo a change in optical properties when their relative positions change and that change can be monitored remotely by optical means. A system and method is also described for detecting glucose that uses such a fusion protein in a variety of formats including subcutaneously and in a bioreactor.

U.S. Pat. No. 5,998,204 to Tsien et al., describes fluorescent protein sensors for detection of analytes. Fluorescent indicators including a binding protein moiety, a donor fluorescent protein moiety, and an acceptor fluorescent protein moiety are described. The binding protein moiety has an analyte-binding region which binds an analyte and causes the indicator to change conformation upon exposure to the analyte. The donor moiety and the acceptor moiety change position relative to each other when the analyte binds to the analyte-binding region. The donor moiety and the acceptor moiety exhibit fluorescence resonance energy transfer when the donor moiety is excited and the distance between the donor moiety and the acceptor moiety is small. The indicators can be used to measure analyte concentrations in samples, such as calcium ion concentrations in cells.

An article by Olesberg J T et al., “Optical microsensor for continuous glucose measurements in interstitial fluid,” Optical Diagnostics and Sensing VI, Proc. of SPIE Vol. 6094, 609403, pp. 1605-7422 (2006), describes an optical glucose microsensor based on absorption spectroscopy in interstitial fluid that can potentially be implanted to provide continuous glucose readings. Light from a GaInAsSb LED in the 2.2-2.4 um wavelength range is passed through a sample of interstitial fluid and a linear tunable filter before being detected by an uncooled, 32-element GaInAsSb detector array. Spectral resolution is provided by the linear tunable filter, which has a 10 nm band pass and a center wavelength that varies from 2.18-2.38 um (4600-4200 cm̂−1) over the length of the detector array. The sensor assembly is a monolithic design requiring no coupling optics. In the present system, the LED running with 100 mA of drive current delivers 20 nW of power to each of the detector pixels, which have a noise-equivalent-power of 3 pW/Hẑ(1/2). This is sufficient to provide a signal-to-noise ratio of 4500 Hẑ(1/2) under detector-noise limited conditions. This signal-to-noise ratio corresponds to a spectral noise level less than 10 uAU for a five minute integration, which is described as being sufficient for sub-millimolar glucose detection.

An article by Klueh U. et al., entitled, “Enhancement of implantable glucose sensor function in vivo using gene transfer-induced neovascularization,” Biomaterials, April, 2005, 26(10):1155-63, states that the in vivo failure of implantable glucose sensors is thought to be largely the result of inflammation and fibrosis-induced vessel regression at sites of sensor implantation. To determine whether increased vessel density at sites of sensor implantation would enhance sensor function, cells genetically engineered to over-express the angiogenic factor (AF) vascular endothelial cell growth factor (VEGF) were incorporated into an ex ova chicken embryo chorioallantoic membrane (CAM)-glucose sensor model. The VEGF-producing cells were delivered to sites of glucose sensor implantation on the CAM using a tissue-interactive fibrin bio-hydrogel as a cell support and activation matrix. This VEGF-cell-fibrin system induced significant neovascularization surrounding the implanted sensor, and significantly enhanced the glucose sensor function in vivo.

The following patents and patent applications may be of interest:

PCT Publication WO 01/50983 to Bloch et al.

PCT Publication WO 01/50983 to Vardi et al., and U.S. patent application Ser. No. 10/466,069 in the national phase thereof

PCT Publication WO 04/028358 to Caduff et al.

PCT Publication WO 05/053523 to Caduff et al.

PCT Publication WO 06/097933 to Bitton et al.

PCT Publication WO 08/018079 to Goldberg et al.

PCT Publication WO 90/15526 to Kertz

U.S. Pat. No. 4,402,694 to Ash et al.

U.S. Pat. Nos. 4,981,779 and 5,001,054 to Wagner

U.S. Pat. No. 5,011,472 to Aebischer et al.

U.S. Pat. No. 5,089,697 to Prohaska

U.S. Pat. No. 5,116,494 to Chick et al.

U.S. Pat. No. 5,443,508 to Giampapa

U.S. Pat. No. 5,529,066 to Palti

U.S. Pat. No. 5,614,378 to Yang et al.

U.S. Pat. No. 5,702,444 to Struthers et al.

U.S. Pat. No. 5,741,334 to Mullon et al.

U.S. Pat. No. 5,834,005 to Usala

U.S. Pat. No. 5,855,613 to Antanavich et al.

U.S. Pat. No. 6,091,974 to Palti

U.S. Pat. No. 6,400,974 to Lesho

U.S. Pat. No. 6,630,154 to Fraker et al.

U.S. Pat. No. 6,846,288 to Nagar et al.

U.S. Pat. No. 7,068,867 to Adoram et al.

U.S. Pat. No. 7,184,810 to Caduff et al.

US Patent Application 2002/0038083 to Houben and Larik

United States Patent Application Publication 2003/0232370 to Trifiro

US Patent Application Publication 2003/0134346 to Amiss et al.

The following articles may be of interest:

Yu-Lung L et al., “A polarimetric glucose sensor using a liquid-crystal polarization modulator driven by a sinusoidal signal,” Optics Communications 259(1), pp. 40-48 (2006)

McNichols J et al., “Development of a non-invasive polarimetric glucose sensor,” IEEE-LEOS Newsletter, 12:30-31 (1998)

Cote C L “Noninvasive and minimally-invasive optical monitoring technologies,” The Journal of Nutrition 131:1596S-1604S (2001)

Wan Q, “Dual wavelength polarimetry for monitoring glucose in the presence of varying birefringence,” A thesis submitted to the Office of Graduate Studies of Texas A&M University (2004)

Olesberg J T, “Noninvasive blood glucose monitoring in the 2.0-2.5 μm wavelength range,” Lasers and Electro-Optics Society. LEOS 2001. The 14th Annual Meeting of the IEEE. Volume: 2, p. 529

Olesberg J T et al., “Tunable Laser Diode System for Noninvasive Blood Glucose Measurements,” Appl. Spectrosc. 59, pp. 1480-1484 (2005)

Olesberg J T et al., “In vivo near-infrared spectroscopy of rat skin tissue with varying blood glucose levels,” Analytical Chemistry 78, pp. 215-223 (2006)

Amir O et al., “Accurate home and clinical use of a non-invasive continuous glucose monitor,” (2006)

Dvir D et al., “Non invasive blood glucose monitoring in the critically ill patients,” European Society for Clinical Nutrition and Metabolism Congress, Istanbul (2006)

Primack H, “Non-invasive sensing of glucose and hemoglobin,” Optical Imaging (2006)

Amir O et al., “Evaluation of a non-invasive continuous glucose monitoring device in a home use setting,” European Association for the Study of Diabetes, 42nd Annual Meeting, Copenhagen-Malmoe, Denmark-Sweden (2006)

Amir O et al., “Highly accurate non-invasive continuous glucose monitoring in clinical and home use settings,” American Diabetes Association, 66th Scientific Session, Washington, D.C. (2006)

Berrebi A et al., “A non-invasive evaluation of hematocrit with a new optical sensor,” European Hematology Association, 11th Congress, Amstaerdam (206)

Kononenko A et al., “Evaluation of a non-invasive blood glucose monitoring device for critically ill patients,” 26th International Symposium on Intensive Care and Emergency Medicine, Brussels (2006)

Primack H, “Non-invasive optical sensing of blood hemoglobin and glucose,” Photonic West, San Jose, Calif. (2006)

Ye K et al., “Genetic engineering of an allosterically based glucose indicator protein for continuous glucose monitoring by fluorescence resonance energy transfer,” Analytical Chemistry, 2003, 75(14), 3451-3459

Tolosa L et al., “Glucose sensor for low-cost lifetime-based sensing using a genetically engineered protein,” Analytical Biochemistry, 267, 114-120 (1999)

Hellinga H et al., “Protein engineering and the development of generic biosensors,” Tibtech, Volume 16 (April, 1998)

Fehr M et al., “In vivo imaging of the dynamics of glucose uptake in the cytosol of COS-7 cells by fluorescent nanosensors,” J. Biol. Chem., 278(21):19127-19133 (2003)

Ackland-Berglund, C et al., “Efficacy of tetracycline-controlled gene expression is influenced by cell type,” BioTechniques 18, 196-200 (1995)

Fillat C et al., “Suicide gene therapy mediated by the herpes simplex virus thymidine kinase gene/ganciclovir system: Fifteen years of application,” Current Gene Therapy, 3(1), pp. 13-26, (February, 2003)

Scognamiglio V et al., “Protein-based biosensors for diabetic patients,” Journal of Fluorescence, 14(5), 491-498 (September, 2004)

Moschou E et al., “Fluorescence glucose detection: Advances toward the ideal in vivo biosensor,” Journal of Fluorescence, 14(5), 535-547 (September, 2004)

Reszka R et al., “Liposome-mediated suicide gene therapy in humans,” Methods in Enzymology, 391, 200-208 (2005)

Deuschle K et al., “Construction and optimization of a family of genetically encoded metabolite sensors by semirational protein engineering,” Protein Sci. 14: 2304-2314 (2005)

Liua L et al., “Glucose permeable poly(dimethyl siloxane) poly(N-isopropyl acrylamide) interpenetrating networks as ophthalmic biomaterials,” Biomaterials Volume 26, Issue 3 pp. 233-244 (2005)

Yonzon C R et al., “A glucose biosensor based on surface-enhanced Raman scattering: Improved partition layer, temporal stability, reversibility, and resistance to serum protein interference,” Anal. Chem., 76 (1), pp. 78-85 (2004)

Koo T W et al., “Measurement of glucose in human blood serum using Raman spectroscopy”, IEEE-LEOS Newsletter 12(2) 18 (1998).

Yokota M et al., “A compact polarimetric glucose sensor using a high-performance fibre-optic Faraday rotator,” Meas. Sci. Technol. 15 pp. 143-147 (2004)

### SUMMARY OF THE INVENTION

In some embodiments of the present invention, a support, e.g., a housing or a scaffold, is configured to be implanted within a body of a subject and is coupled to (a) a sampling region, e.g., a chamber, configured to passively allow passage therethrough of a fluid from a subject, and (b) an optical measuring device which measures a parameter of the fluid in the chamber. Typically, the housing is subcutaneously implanted within the subject. Typically, the optical measuring device is configured to measure a concentration of an analyte, e.g., glucose, in interstitial fluid of the subject. The optical measuring device typically comprises a light source, i.e., a system providing visible or non-visible light, and a detecting system. The support provides a sampling region which is typically disposed in optical communication with the light source and the detecting system.

In some embodiments, the support comprises and/or is coupled to an optically-transparent and glucose-permeable material, e.g., a gel or polymer, configured to define the sampling region of the support.

In some embodiments, the sampling region is surrounded by a selectively-permeable membrane which restricts passage therethrough of substances, e.g., cells, which could potentially interfere with the measuring of the parameter of the fluid. The membrane may be present in the support independently of or in combination with the transparent glucose-permeable material. Typically, the membrane is configured to restrict passage, into the sampling region, of cells and molecules having a molecular weight greater than the molecular weight of the analyte configured to be measured by the device. In some embodiments, the sampling region comprises cells engineered to produce a protein that is able to bind with the analyte and to undergo a conformational change in a detectable manner. An implanted device detects the conformational change, and, in response, generates a signal indicative of a level of the analyte in the subject. Typically, but not necessarily, FRET techniques known in the art are used to detect the conformational change. These genetically-engineered cells may be used in combination with the detection methods described hereinbelow.

In either embodiment, the concentration of the analyte is measured using polarimetric techniques which measure the concentration of the analyte according to the polarization of light that passes from the light source and through the sampling region. In such an embodiment, polarizing filters are disposed in optical communication with the light source and/or the detecting system. Alternatively or additionally, the concentration of the analyte is measured using absorbance spectroscopy techniques. In such an embodiment, the absorbance spectroscopy is used to directly measure the concentration of the analyte in the sampling region. Alternatively or additionally, the absorbance spectroscopy device comprises a plurality of detectors configured to detect optical scattering of the illuminated light (the scattering being induced by the presence of the analyte in the fluid). The plurality of detectors are configured to increase the signal-to-noise ratio.

Techniques described herein to optically measure the concentration of the analyte in the fluid typically use LEDs, solid-state lasers, or laser diodes as the light source, and photodetectors, e.g., linear detector arrays, as the detecting system.

In some embodiments of the present invention, the device comprises at least one mirror disposed in the optical path between the light source and the detecting system. The mirror is configured to lengthen the optical path of the light emitted from the light source.

In some embodiments of the present invention, the support comprises an annular ring scaffold having a wall thereof. The annular ring defines a substantially disc-shaped sampling region and houses a plurality of light sources and detecting systems. For some applications, the annular ring has an upper surface and a lower surface that are each coupled to a respective selectively-permeable membrane which restricts passage of cells into the sampling region. In some embodiments, the scaffold houses the glucose-permeable and optically-transparent material in combination with the respective membranes. Alternatively, the selectively-permeable membranes are not provided, and the glucose-permeable and optically-transparent material generally provides the functionality of the membranes. Typically, the upper surface and/or the lower surface of the disc-shaped sampling region provide a large surface area for passive fluid transport through the sampling region. Typically, the combined surface area provided for substance transport by the upper and lower surfaces of the sampling region is at least 50% (e.g., at least 70%) of a total surface area of the optical measuring device.

In some embodiments of the present invention, the sampling region is disposed remotely from the optical measuring device. For example, the sampling region may be disposed within a blood vessel, e.g., a vena cava, of the subject, while the optical measuring device is disposed outside of the blood vessel. In some embodiments, the optical measuring device is disposed outside the body of the subject. Alternatively, the optical measuring device is disposed inside the body of the subject in a vicinity of the blood vessel and is coupled to the sampling region by optical fibers. In such an embodiment, the optical measuring device measures a concentration of an analyte in the blood of the subject.

There is therefore provided, in accordance with an embodiment of the present invention, apparatus, including:

a support configured to be implanted within a body of a subject;

a sampling region coupled to the support, the apparatus configured to passively allow passage through the sampling region of at least a portion of fluid from the subject; and

an optical measuring device in optical communication with the sampling region, including:
at least one light source configured to transmit light through at least a portion of the fluid, and
at least one sensor configured to measure a parameter of the fluid by detecting light passing through the fluid.

In an embodiment, the sampling region has at least one surface thereof configured for the passage of the portion of fluid therethrough, the surface having a surface area that is at least 50% of a total surface area of the apparatus.

In an embodiment, the sampling region has at least one surface thereof configured for the passage of the portion of fluid therethrough, the surface having a surface area that is at least 70% of a total surface area of the apparatus.

In an embodiment, the portion of the fluid includes glucose, and the apparatus is configured to passively allow passage of the glucose through the sampling region.

In an embodiment, the parameter of the fluid includes glucose concentration, and the optical measuring device is configured to measure a concentration of glucose in the fluid.

In an embodiment, the apparatus is configured for subcutaneous implantation within the subject.

In an embodiment, the light source includes a light emitting diode.

In an embodiment, the light source is configured to emit infrared light.

In an embodiment, the apparatus includes a transmitter and a receiver, the transmitter configured to be in communication with the sensor, and the receiver configured to be disposed at a site outside the body of the subject, and the transmitter is configured to transmit the measured parameter to the receiver.

In an embodiment, the apparatus includes a drug administration unit configured to administer a drug in response to the measured parameter.

In an embodiment, the sampling region has a length between 1 mm and 10 mm.

In an embodiment, the sampling region has a length between 10 mm and 100 mm.

In an embodiment, the apparatus is configured to measure the parameter of the fluid using absorbance spectroscopy.

In an embodiment, the sensor is configured to measure the light scattered within the sampling region.

In an embodiment, the apparatus includes a housing coupled to the support and configured to surround the sampling region, the housing having at least one opening formed therein configured for passage of the fluid therethrough and into the housing.

In an embodiment, the light source and the sensor are physically separated by at least a portion of the sampling region.

In an embodiment, the apparatus includes cells disposed within the sampling region, the cells being genetically engineered to produce, in situ, a protein configured to measure the parameter of the fluid.

In an embodiment, the support is configured for implantation within a blood vessel of the subject, and the optical measuring device is configured to be in optical communication with a vicinity of the blood vessel in which the support is implanted.

In an embodiment, the blood vessel includes a vena cava of the subject, and the support is configured for implantation within the vena cava of the subject.

In an embodiment, the support is shaped to define a cylindrical support, and the sampling region is disposed within the wall of the cylindrical support.

In an embodiment, the support includes a disc-shaped support.

In an embodiment, the support is shaped to define a cylindrical support, the cylindrical support defining a lumen thereof that surrounds the sampling region.

In an embodiment, the apparatus includes cells disposed within the sampling region, the cells being genetically engineered to produce, in situ, a protein configured to facilitate a measurement of the parameter of the blood.

In an embodiment:

the support is shaped to define a cylindrical support defining a lumen thereof,

the sampling region is disposed within the lumen, and

the cells are genetically engineered to secrete the protein into the sampling region.

In an embodiment, the optical measuring device is configured to be disposed externally to the blood vessel.

In an embodiment, the apparatus includes at least one optical fiber, the optical fiber is coupled at a first end to the optical measuring device, and at a second end to the support, and light from the light source is provided to the sampling region via the optical fiber.

In an embodiment, the fluid includes components of blood of the subject, and the apparatus is configured to facilitate a measurement of a parameter of the blood of the subject.

In an embodiment, the parameter of the blood includes a level of glucose in the blood, and the apparatus is configured to facilitate a measurement of the level of glucose in the blood of the subject.

In an embodiment, the sensor is configured to measure the parameter by detecting a photoacoustic effect induced by the light passing through the fluid.

In an embodiment, the light source includes a solid-state laser.

In an embodiment, the light source is configured to emit visible light.

In an embodiment, the sensor includes a photodetector.

In an embodiment, the light source includes a plurality of light sources, and the sensor includes a plurality of photodetectors.

In an embodiment, the light source is configured to emit polarized light, and the apparatus further includes at least one first polarizing filter having an orientation thereof and configured to filter the polarized light emitted from the light source into the sampling region.

In an embodiment, the apparatus includes at least one second polarizing filter configured to filter to the sensor the polarized light passing through the sampling region.

In an embodiment, the second polarizing filter has an orientation thereof that is substantially perpendicular to the orientation of the first polarizing filter.

In an embodiment, the light includes visible light, and the apparatus further includes a tunable filter configured to refract the light emitted from the light source into a plurality of monochromatic bands.

In an embodiment, the tunable filter includes a Faraday rotator.

In an embodiment, the sensor includes a plurality of photodetectors, each photodetector detecting a respective one of the plurality of monochromatic bands.

In an embodiment, the sampling region includes a permeable material selected from the group consisting of: agarose, silicone, polyethylene glycol, gelatin, an optical fiber capillary, a polymer, a co-polymer, and an alginate, the permeable material being positioned to passively allow passage therethrough of the portion of fluid in the sampling region.

In an embodiment, the material includes an optically-transparent and glucose-permeable material.

In an embodiment, the material is configured to restrict passage of cells into the sampling region.

In an embodiment, the sampling region includes a gel including extracellular matrix and a permeable material selected from the group consisting of: agarose, silicone, polyethylene glycol, gelatin, an optical fiber capillary, a polymer, a co-polymer, and an alginate.

In an embodiment, the gel includes an optically-transparent and glucose-permeable gel.

In an embodiment, the gel is configured to restrict passage of cells into the sampling region.

In an embodiment, the apparatus includes a selectively-permeable membrane coupled to the support, the membrane being configured to surround the sampling region.

In an embodiment, the fluid includes interstitial fluid, and the membrane is configured to restrict passage therethrough of cells.

In an embodiment, the apparatus includes at least one reflector, configured to reflect to the sensor light emitted from the light source that has passed through the sampling region.

In an embodiment, the at least one reflector includes a plurality of reflectors, each one of the plurality of reflectors is disposed at a respective location with respect to the sampling region, and the plurality of reflectors is configured to lengthen an optical path between the light source and the sensor.

In an embodiment, the support includes a disc-shaped housing, and the sampling region includes a disc-shaped sampling region.

In an embodiment, the sampling region has at least one surface thereof configured for the passage of the portion of fluid therethrough, the surface having a surface area that is at least 50% of a total surface area of the apparatus.

In an embodiment, the sampling region has at least one surface thereof configured for the passage of the portion of fluid therethrough, the surface having a surface area that is at least 70% of a total surface area of the apparatus.

In an embodiment:

the support is shaped to define a wall thereof surrounding the sampling region,

the at least one light source includes a plurality of light sources disposed along the wall of the support and configured to transmit light through the sampling region, and

the at least one sensor includes a plurality of sensors disposed along the wall of the support and configured to receive at least a portion of the light passing through the fluid.

In an embodiment, the support has a first surface and a second surface, and the apparatus further includes a first selectively-permeable membrane coupled to the first surface and a second selectively permeable membrane coupled to the second surface.

In an embodiment, the first and second selectively-permeable membranes are configured to restrict passage of cells therethrough.

There is additionally provided, in accordance with an embodiment of the present invention, a method for detecting a parameter of a fluid, including:

implanting within a body of a subject a support coupled to a sampling region configured to passively allow passage therethrough of at least a portion of the fluid;

restricting passage of cells into the sampling region;

transmitting light through the portion of the fluid; and

in conjunction with the transmitting, measuring the parameter of the fluid by detecting passage of light through the fluid.

There is also provided, in accordance with an embodiment of the present invention, apparatus, including:

a support configured to be implanted within a body of a subject;

at least one membrane coupled to the support configured to define a sampling region, the membrane configured to passively allow passage through the sampling region of a fluid from the subject; and

an optical measuring device in optical communication with the sampling region, including:
at least one light source configured to transmit light through at least a portion of the fluid, and
at least one sensor configured to measure a parameter of the fluid by detecting light that has passed through the fluid.

In an embodiment:

the support is shaped to define an annular wall thereof surrounding the sampling region,

the at least one light source includes a plurality of light sources disposed along the wall of the support and configured to transmit light through the sampling region, and

the at least one sensor includes a plurality of sensors disposed along the wall of the support and configured to receive at least a portion of the light passing through the fluid.

In an embodiment, the support has a first surface and a second surface, and the at least one membrane includes a first selectively-permeable membrane coupled to the first surface and a second selectively-permeable membrane coupled to the second surface.

In an embodiment, the first and second selectively-permeable membranes are configured to restrict passage of cells therethrough.

The present invention will be more fully understood from the following detailed description of embodiments thereof, taken together with the drawings, in which:

### BRIEF DESCRIPTION OF THE DRAWINGS

FIG. 1 is a schematic illustration of an optical measuring device, in accordance with an embodiment of the present invention;

FIGS. 2 and 3 are schematic illustrations of the optical measuring device of FIG. 1, in accordance with respective embodiments of the present invention;

FIG. 4 is a schematic illustration of an optical measuring device, in accordance with another embodiment of the present invention;

FIG. 5 is a schematic illustration of the optical measuring device of FIG. 4, in accordance with an embodiment of the present invention;

FIG. 6 is a schematic illustration of the optical measuring device comprising genetically-engineered cells, in accordance with an embodiment of the present invention;

FIG. 7 is a schematic illustration of the optical measuring device, in accordance with still another embodiment of the present invention;

FIG. 8 is a schematic illustration of a disc-shaped optical measuring device, in accordance with an embodiment of the present invention; and

FIG. 9 is a schematic illustration of a sampling region disposed within a blood vessel of the subject, in accordance with an embodiment of the present invention.

### DETAILED DESCRIPTION OF THE EMBODIMENTS

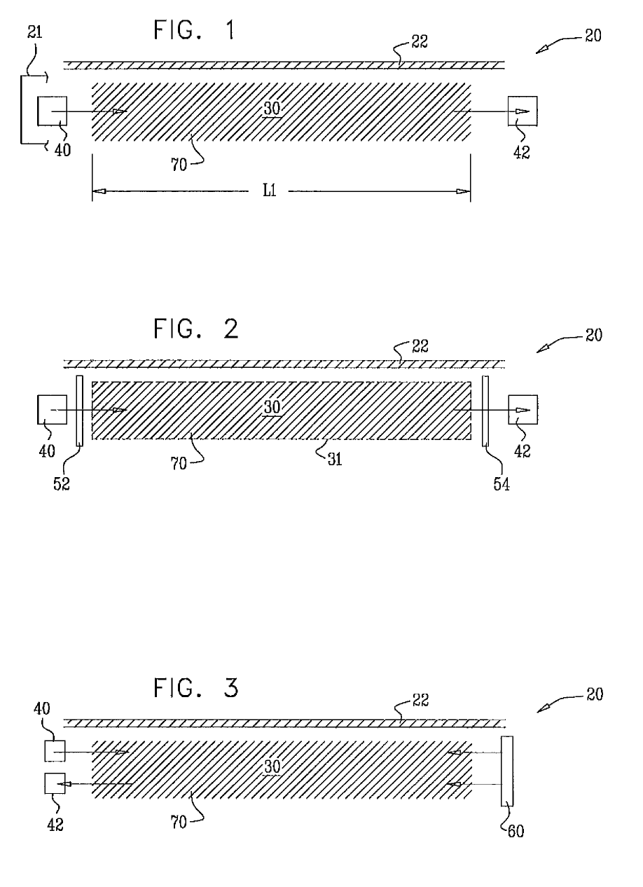

Reference is now made to FIG. 1, which is a schematic illustration of an optical measuring device 20 comprising an electromagnetic light source 40 and a detecting system 42, in accordance with an embodiment of the present invention. Typically, light source 40 is configured to emit electromagnetic radiation that is in the visible or infrared range. Optical measuring device 20 is configured to detect and measure a concentration of an analyte, e.g., glucose, in interstitial fluid of a subject. (In the context of the specification, examples of the analyte being glucose are by way of illustration and not limitation.) Typically, device 20 is designated for subcutaneous implantation under skin 22 of a subject. Typically, device 20 comprises a support 21 (e.g., a housing, a scaffold, or glue). A sampling region 30 is disposed within an area defined by support 21 of device 20, typically between light source 40 and detecting system 42 (as shown). Support 21 is configured to facilitate proper spatial relationship between sampling region 30, source 40, and detecting system 42.

In some embodiments, light source 40 comprises any suitable light source, e.g., a light emitting diode (LED), an organic light emitting diode (OLED), a laser diode, or a solid-state laser.

In some embodiments, support 21 comprises a selectively-permeable membrane, and support 21 is coupled to the selectively-permeable membrane. In some embodiments, the membrane is optically transparent. Typically, the membrane is permeable to molecules having a molecular weight equal to or less than the molecular weight of the analyte (e.g., glucose) configured to be measured by device 20. Typically, the membrane is configured to restrict passage into sampling region 30 of cells from outside device 20.

Sampling region 30 comprises an optically-transparent and glucose-permeable material 70. In some embodiments, material 70 comprises, by way of illustration and not limitation, alginate, agarose, silicone, a polymer, a co-polymer polyethylene glycol (PEG), and/or gelatin. Alternatively or additionally, material 70 comprises a glucose-permeable gel comprising extracellular matrix (ECM) in combination with one or more of the above-listed optically-transparent and glucose-permeable materials. For some applications, material 70 comprises an optically-transparent and glucose-permeable copolymer, e.g., Poly(dimethyl siloxane) (PDMS), Poly(N-isopropyl acrylamide) (PNIPAAM), and other optically-transparent and glucose-permeable copolymers, or other copolymers known in the art. In some embodiments, material 70 comprises a plurality of hollow capillary fibers configured for optical transmission of the light from source 40 and to allow for passage of certain constituents (e.g., small molecules such as glucose) of fluid through sampling region 30 in order to facilitate optical measuring of the analyte in region 30.

Typically, material 70 is configured to passively allow passage therethrough of certain constituents (e.g., small molecules such as glucose) of the interstitial fluid of the subject that have a molecular weight smaller than the desired molecular weight cutoff defined by material 70. For example, the molecular weight cutoff allows passage therethrough of glucose molecules present in the interstitial fluid. In some embodiments, the molecular weight cutoff allows passage through material 70 of only glucose molecules present in the interstitial fluid and of other molecules having a molecular weight equal to or less than the molecular weight of the glucose molecule. That is, material 70 is configured to restrict passage therethrough into sampling region 30 of molecules having a molecular weight greater than a molecular weight of a glucose molecule.

Material 70 defining sampling region 30 has suitable fixed dimensions such that the glucose concentration within region 30 is in equilibrium with glucose concentrations of adjacent interstitial fluid not within region 30. The fixed dimensions of region 30 enable passage therethrough of a definite, consistent, relatively small volume, e.g., up to 1 mL, of fluid during each measurement. In some embodiments, sampling region 30 enables passage therethrough of a volume between 0.01 mL and 1 mL, e.g., between 0.05 mL and 0.5 mL, of fluid during each measurement. Sampling region 30 has a length L1 of between 1 mm and 100 mm.

Because of the typically small size of region 30, the concentration of the analyte outside of device 20 is in general equilibrium with the average concentration of analyte in the fluid measured in region 30 during measurements thereof. Therefore, measuring the concentration of glucose in sampling region 30 provides an indication of the concentration of glucose in the body of the subject.

Typically, material 70 has a relative refractive index of about 1.35-1.40, which prevents or minimizes loss of light and refraction thereof.

Material 70 permits passage therethrough of the constituents of the interstitial fluid having a molecular weight smaller than the molecular weight cutoff defined by material 70. Typically, material 70 restricts passage of cells from outside device 20 and into sampling region 30. This permeability typically does not affect the equilibrium of glucose concentrations between the fluid inside sampling region 30 and the interstitial fluid not inside device 20.

Typically, light source 40 transmits light through sampling region 30 and toward detecting system 42 in the direction indicated by the arrows in the figure. For embodiments in which source 40 emits polarized light, the light is rotated by the glucose in sampling region 30. Detecting system 42 typically comprises a sensor (e.g., a photodetector such as a linear detector) for detecting the rotation of the light that has passed through region 30. In some embodiments, detecting system 42 comprises an array of sensors.

A control unit, e.g., a microprocessor (not shown), is typically in communication with device 20 and facilitates real-time quantitative analysis of glucose in sampling region 30. Typically, the control unit drives light source 40 to emit light within region 30 in accordance with various emission parameters, such as duty cycle (e.g., number and/or timing of hours of operation per day), wavelengths, and amplitudes. In some embodiments, light source 40 is actuated by the control unit using a duty cycle of less than 0.02% (e.g., on for 10 msec, off for 1 minute). Alternatively, the control unit is configured to drive light source 40 to emit light with a different duty cycle, or continuously. In some embodiments, the control unit is externally programmable following implantation to allow calibration or intermittent optimization of the various emission parameters of light source 40. A power source (not shown) is coupled to device 20 and is configured to supply power thereto.

In some embodiments, the control unit of device 20 is coupled to a drug administration unit (not shown) configured to administer a drug in response to the measured parameter, e.g., in response to the level of glucose measured in the interstitial fluid. In some embodiments, the drug administration unit comprises an insulin pump, which supplies insulin or another drug to the body in response to the level of the analyte determined by device 20.

Reference is now made to FIG. 2, which is a schematic illustration of device 20 as described hereinabove with reference to FIG. 1, with the exception that device 20 comprises optical filters 52 and 54, in accordance with an embodiment of the present invention. Typically, optical measuring device 20 is configured to measure the concentration of glucose in sampling region 30 using at least one technique such as polarimetry, absorbance spectroscopy, and/or other optical measuring techniques known in the art.

It is to be noted that although device 20 is shown comprising two filters 52 and 54, device 20 may comprise one, both, or neither of filters 52 and 54. For some embodiments in which filters 52 and 54 are polarizing filters, filter 54 is disposed substantially perpendicularly with respect to filter 52.

As shown, a selectively-permeable, biocompatible membrane 31 is disposed around region 30, and is configured to restrict passage of cells into sampling region 30. In some embodiments, membrane 31 comprises a hydrophobic membrane, e.g., a nitrocellulose membrane. In some embodiments, membrane 31 comprises a polyvinylidene difluoride, or PVDF, membrane. In some embodiments membrane 31 has a molecular weight cutoff of around 500 kDa. It is to be noted, however, that embodiments described herein may be implemented independently of membrane 31.

Membrane 31 provides permeability for passage therethrough of certain constituents (e.g., small molecules such as glucose) of the interstitial fluid that have a molecular weight smaller than the molecular weight cutoff defined by membrane 31. Typically, membrane 31 allows the passage into sampling region 30 of molecules having a molecular weight below a desired cut-off. For example, the molecular weight cutoff allows passage through membrane 31 of only glucose molecules present in the interstitial fluid and of other molecules having a molecular weight less than or generally equal to the molecular weight of the glucose molecule. That is, membrane 31 is configured to restrict passage therethrough into sampling region 30 of molecules or other body fluid components having a molecular or characteristic weight substantially greater than the molecular weight of a glucose molecule, e.g., cells.

This permeability typically does not affect the equilibrium of glucose concentrations between the fluid inside the device and the interstitial fluid not inside the device, as described hereinabove with respect to material 70 with reference to FIG. 1.

In some embodiments, membrane 31 is optically transparent. Typically, membrane 31 is permeable to molecules having a molecular weight less than or generally equal to the molecular or characteristic weight of the analyte (e.g., typically, glucose) that is measured by device 20. Typically, membrane 31 restricts passage into sampling region 30 of cells from outside device 20.

Membrane 31 defines sampling region 30 and has suitable fixed dimensions such that the glucose concentration within region 30 is in general equilibrium with glucose concentrations of the interstitial fluid not within region 30, as described hereinabove with reference to material 70 (with reference to FIG. 1). The fixed dimensions of region 30 enable passage therethrough of a definite, consistent, relatively small volume, e.g., up to about 1 mL, of fluid during each measurement.

It is to be noted that membrane 31 is shown by way of illustration and not limitation. For example, material 70 of sampling region 30 may be disposed within an area defined by a support (as described hereinabove with reference to FIG. 1) and/or upon a scaffold independently of or in combination with membrane 31. The scaffold may comprise a porous material configured to allow passage therethrough into region 30 of constituents of the interstitial fluid having a molecular weight smaller than the desired molecular weight cutoff defined by the scaffold. Typically, the scaffold is configured to restrict passage of cells into sampling region 30. The scaffold may be used independently of or in combination with membrane 31.

In the following techniques, device 20 comprises filters 52 and 54 in order to facilitate detection of glucose concentration using polarimetry techniques known in the art. Polarimetry techniques described herein are typically practiced in combination with polarimetry techniques described in one or more of the references in the Background section of the present patent application, namely:
U.S. Pat. No. 5,209,231,
U.S. Pat. No. 6,188,477;
U.S. Pat. No. 6,577,393;
Wan Q, “Dual wavelength polarimetry for monitoring glucose in the presence of varying birefringence,” A thesis submitted to the Office of Graduate Studies of Texas A&M University (2004); and
Yu-Lung L et al., “A polarimetric glucose sensor using a liquid-crystal polarization modulator driven by a sinusoidal signal,” Optics Communications 259(1), pp. 40-48 (2006).

All of these references are incorporated herein by reference.

Typically, light emitted from light source 40 includes wavelengths within the visible range. In such an embodiment, light source 40 comprises an incandescent light bulb, by way of illustration and not limitation, and the linear polarization vector of light rotates when the light is passed from light source 40 through region 70 comprising a chiral analyte, e.g., glucose. The rotation measured is proportional to the concentration of the glucose being monitored.

In addition to the dependence of the rotation of the linear vector of the polarized light on the concentration of the chiral analyte, the amount of the rotation of the linear vector of the polarized light also depends on (1) the properties, e.g., optical path length, defined by region 30, and (2) the wavelength of the light used for the measurement. The relationship between a degree of optical rotation and the concentration of glucose in region 30, in addition to parameters of region 30, is expressed in the following equation:

Phi=(alpha.sub.lambda)LC, (eq. 1)

where phi is the angle of rotation, alpha.sub.lambda is the specific rotation at a wavelength lambda, L is the path length, and C is the concentration of glucose in region 30. Due to the dependency of concentration measurement upon the optical path length provided by sampling region 30, material 70 is optically-transparent and glucose-permeable, and has a particular length. Additionally, the physical length of sampling region 30 can be reduced while still maintaining a desired optical path length. For example, region 30 may have a physical length that is shorter than the optical path length of the light emitted from source 40. That is, region 30 may comprise at least one mirror configured to reflect the light and thereby lengthen the optical path length. In some embodiments, the mirror may be disposed externally to region 30 and in communication therewith.

In some embodiments, region 30 may comprise at least one hollow optical waveguide, e.g., in the form of a light-conductive capillary fiber, in order to elongate the optical path of the light emitted from light source 40. For embodiments in which one waveguide is used, the waveguide may be wound or bundled in order to elongate the optical path of the light emitted from light source 40. In such an embodiment, the analyte passes through the hollow waveguide.

Subcutaneous implantation of device 20 prevents any unwanted depolarization that is typically caused by transmitting a beam of polarized light through skin 22 of the subject, because the thickness of skin 22 typically provides a small optical path length and generates a relatively low signal-to-noise ratio. The subcutaneous positioning of device 20 in combination with the characteristics of regions 30 (described hereinabove) enables light to be passed from source 40, and through region 30, without substantial depolarization of the light.

In order to ensure that the rotated light will pass through filter 54, device 20 provides a suitable length of region 30 that is typically between 1 mm and 10 mm, or between 10 mm and 100 mm.

It is to be noted that region 30 may have any suitable optical path length in accordance with a level of specificity and sensitivity of device 20, typically, 10 mm. Increasing the optical path length provided by region 30 increases the sensitivity of device 20.

For embodiments in which the level of glucose is measured using polarimetric techniques described herein, device 20 comprises a beam-splitter configured to split the beam from light source 40 into two or more beams, e.g., a principle beam and a reference beam. In some embodiments, at least one reference beam, e.g., two reference beams, are created, and the reference beam(s) passes through a polarized filter. In some embodiments, the principle beam and the reference beam have substantially equal optical paths that differ only in factors that support quantitative evaluation of the concentration of the analyte, e.g., glucose, in material 70 within region 30. For example: (a) sampling region 30 may provide a portion of material 70 that is configured to not contain fluid, and (b) the reference beam may be directed through the portion of region 30 that contains material 70 without the analyte, while (c) the principle beam passes in parallel with the reference beam through a portion of region 30 that contains material 70 with the analyte. In these embodiments, the rotation induced by the analyte in material 70 is quantified as the difference between the rotation of the principle beam and the rotation of the reference.

In some embodiments, the reference beam is not polarized, while the principle beam is polarized. In such an embodiment, the reference beam serves as a control for estimating the portion of decrease in measured intensity of the polarized principle beam on detector 42 that is not due to polarization.

In some embodiments, light source 40 comprises a monochromatic light emitting diode (LED), and detecting system 42 comprises a single photodetector. The intensity of light incident on the photodetector is proportional to the angle at which the light is rotated in the sample, and is proportional to the concentration of glucose in sampling region 30.

In some embodiments, light source 40 comprises a white light emitting diode (LED) or a broad-band LED. In such an embodiment, filter 52 comprises a filter system having (1) a polarizing filter, and (2) a tunable, linear optical filter which refracts the white light into monochromatic bands, i.e., light having various wavelengths. The filter system is typically regulated by the control unit described hereinabove. The tunable optical filter enables the emission of various wavelengths through region 30. In such an embodiment, increasing the number of wavelengths which measure the same property within region 30 increases the signal-to-noise ratio.

For embodiments in which light source 40 comprises a white LED, filter 52 typically comprises a polarizing filter. Filter 54 comprises a linear, tunable filter which sorts the polarized light from region 30 according to specific wavelength bands. In some embodiments, light source 40 comprises an array of monochromatic LEDs, and each LED is adapted to transmit a specific wavelength through sampling region 30. Typically, every monochromatic LED is actuated simultaneously. Alternatively, the monochromatic LEDs are actuated in succession. In some embodiments, filters 52 and 54 comprise polarizing filters. Detecting system 42 typically comprises a photodetector. Alternatively, detecting system 42 comprises an array of photodetectors, and each photodetector is configured to detect a specific wavelength band that has been emitted from a specific monochromatic LED and has traveled through sampling region 30.

In some embodiments of the present invention, device 20 is configured to measure the glucose concentration using photoacoustic spectroscopy. In such an embodiment, light source 40 comprises at least one laser diode or a solid-state laser, and detecting system 42 comprises at least one acoustic detector. In some embodiments, a single, tunable laser diode is configured to transmit light at variable wavelengths. In some embodiments, light source 40 comprises a plurality of laser diodes. Each of the plurality of laser diodes is configured to emit a specific wavelength detectable by a single acoustic detector of detecting system 42. Alternatively, detecting system 42 comprises an array of detectors, and each detector is configured to detect a specific wavelength. As described hereinabove, filters 52 and 54 may comprise polarizing filters.

In some embodiments, source 40 comprises an array of energy sources, e.g., solid-state laser sources that generate detectable photoacoustic effects. Each laser source of the array emits laser light having a respective wavelength. In such an embodiment, detecting system 42 comprises an acoustic detector. In some embodiments, source 40 comprises a plurality of solid-state lasers, and detecting system 42 comprises a plurality of acoustic detectors.

FIG. 3 shows optical measuring device 20 comprising at least one mirror 60, in accordance with an embodiment of the present invention. Light source 40 is configured to transmit light (as described hereinabove) into sampling region 30. Device 20 is configured to facilitate optical determination of analyte concentration within region 30 using optical methods described herein and other methods known in the art, e.g., polarimetry and/or absorbance spectroscopy. Light is reflected from mirror 60, which extends the optical path of the light within region 30. Extending the optical path of the light thus satisfies the conditions (set forth in equation 1) for optimizing the illumination/detection of analyte concentrations in the region 30. Once the light is reflected from mirror 60, it is absorbed by detecting system 42, as described hereinabove.

Detecting system 42 is shown positioned adjacent to light source 40 and on the same side of region 30 by way of illustration and not limitation. For example, light source 40 and detecting system 42 may be disposed at any suitable location with respect to region 30. Light source 40 and detecting system 42 may be physically separated by at least a portion of sampling region 30. The respective orientations of light source 40 and detecting system 42 are thus governed by the positioning of mirror 60 at any suitable location with respect to sampling region 30.

Sampling region 30 is shown comprising optically-transparent and glucose-permeable material 70 by way of illustration and not limitation. For example, sampling region 30 may comprise a suitable number (e.g., between 1 and 10) of layers of polymers, e.g., polytetrafluoroethylene (PTFE). Additionally, sampling region 30 may be surrounded by selectively-permeable membrane 31, as shown in FIG. 2.

Reference is now made to FIG. 4, which is a schematic illustration of sampling region 30 as described hereinabove with reference to FIG. 1, with the exception that sampling region 30 is surrounded by a housing 32, in accordance with an embodiment of the present invention. In some embodiments, housing 32 is shaped to define a tube, by way of illustration and not limitation. For example, housing 32 may be shaped to define a rectangular housing. As shown, sampling region 30 of housing 32 comprises optically-transparent and glucose-permeable material 70 (as described herein above with reference to FIG. 1) by way of illustration and not limitation. For example, sampling region 30 may be hollow.

Typically, housing 32 is shaped to define a substantially tubular structure having a first opening 35 and a second opening 37 to allow for passage therethrough of certain constituents (e.g., small molecules such as glucose) of interstitial fluid into housing 32. Openings 35 and 37 are shown at the portions of housing 32 that define the two longitudinal ends of housing 32 by way of illustration and not limitation. For example, first and second openings 35 and 37 may be disposed at opposing lateral sides of housing 32 that define the length of housing 32. In some embodiments, openings 35 and 37 are disposed along the entire length of housing 32.

As shown, first opening 35 is disposed at a first end 152 of housing 32 and second opening 37 is disposed at a second end 154 of housing 32. Housing 32 defining sampling region 30 has suitable dimensions such that the glucose concentration within region 30 is generally in equilibrium with glucose concentrations of the interstitial fluid not within region 30.

As shown, respective membranes 31 are coupled to housing 32 at each opening 35 and 37. It is to be noted that housing 32 surrounding material 70 may be used independently of membranes 31.

Typically, due to the dimensions of housing 32, a defined volume of fluid remains within region 30 during one or more measurements of the analyte concentration in the defined volume. Since a consistent volume of fluid remains within region 30 during the one or more measurements, a lag time between successive measurements of the analyte in sampling region 30 is minimized.

It is to be noted that first and second openings 35 and 37 are shown by way of illustration and not limitation. For example, housing 32 may provide only one opening. Additionally, it is to be further noted that housing 32 is shaped to define a substantially tubular structure by way of illustration and not limitation. For example, device 20 may comprise a flat surface that defines sampling region 30.

Although device 20 is shown in FIG. 4 as not comprising filters 52 and 54, it is to be noted that filters 52 and/or 54 described herein may be used in combination with device 20 of FIG. 4.

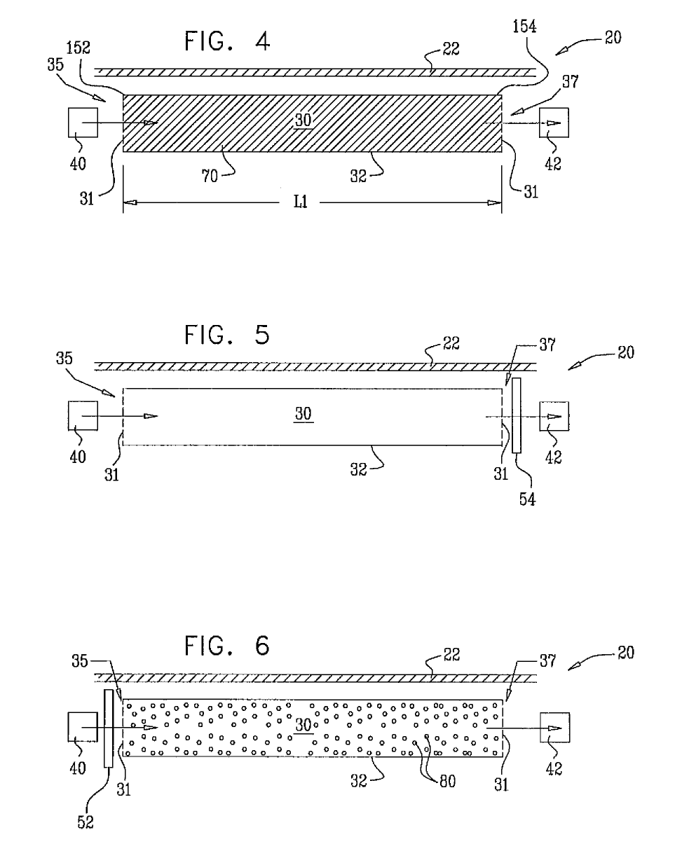

FIG. 5 shows optical measuring device 20 as described hereinabove with reference to FIG. 4, with the exception that device 20 comprises filter 54 and housing 32 that surrounds a hollow sampling region, in accordance with an embodiment of the present invention.

As shown, respective membranes 31 are coupled to housing 32 at each opening 35 and 37.

In some embodiments, light source 40 comprises a solid-state laser. Typically, use of the solid-state laser is configured to facilitate detection of glucose concentration by polarimetry. In such an embodiment, detecting system 42 comprises a photodetector and filter 54 comprises a polarizing filter that is oriented perpendicularly with respect to the polarity of the laser beam as it is emitted from source 40.

Reference is now made to FIG. 6, which is a schematic illustration of device 20 as described hereinabove with reference to FIG. 5, with the exception that sampling region 30 comprises genetically-engineered cells 80 within housing 32, in accordance with an embodiment of the present invention. Cells 80 are genetically engineered to express a protein configured to facilitate optical quantification of the analyte in sampling region 30, e.g., as described in PCT Publication WO 06/006166 to Gross et al., and PCT Publication WO 07/110867 to Gross et al. Cells 80 are engineered to produce a molecule (e.g., a protein) that is able to bind with an analyte and to undergo a conformational change in a detectable manner. Typically, detecting system 42 detects the conformational change, and in response, generates a signal indicative of a level of the analyte in the subject. Typically, but not necessarily, FRET techniques known in the art are used to detect the conformational change.

For embodiments in which FRET is used, cells 80 are genetically engineered to produce, in situ, sensor proteins comprising a fluorescent protein donor (e.g., cyan fluorescent protein (CFP)), a fluorescent protein acceptor (e.g., yellow fluorescent protein (YFP)), and a binding protein (e.g., glucose-galactose binding protein), for the analyte. As appropriate, the sensor proteins may generally reside in the cytoplasm of cells 80 and/or may be targeted to reside on the cell membranes of cells 80, and/or may be secreted by cells 80 into sampling region 30. The sensor proteins are configured such that binding of the analyte to the binding protein changes the conformation of the sensor proteins, and thus the distance between respective donors and acceptors.

In such an embodiment, light source 40 comprises a source of light, e.g., a laser diode, that emits light that is absorbed by the aforementioned fluorescing molecules. Using the signal from light source 40, detecting system 42 detects the spectral changes that result from the change in distance and energy transfer from the donor to the acceptor proteins. The relative quantities of the signal resulting from subsets of the sensor proteins that are in each of the two conformations enable the calculation of the concentration of the analyte.

As shown in FIG. 6, respective selectively-permeable membranes 31 (as described hereinabove with reference to FIG. 2) are disposed at openings 35 and 37. Membranes 31 immunoisolate cells 80 and function to restrict passage of (a) cells from outside device 20 into sampling region 30, and (b) cells 80 from within sampling region 30 to outside device 20. In some embodiments, portions of cells 80 are encapsulated within respective membranes within housing 32.

In some embodiments, cells 80 are immobilized upon a first scaffold polymer. The polymer may be used independently of or in combination with housing 32. For example, housing 32 may surround the polymer. In some embodiments, a second polymer (e.g., optically-transparent and glucose-permeable material 70 described hereinabove with reference to FIG. 1) may be used in combination with the first scaffold polymer.

In some embodiments, cells 80 are not surrounded by housing 32, rather cells 80 are surrounded by a biocompatible selectively-permeable membrane. In some embodiments, the membrane is configured to be optically transparent. Typically, the membrane is permeable to molecules having a molecular or characteristic weight equal to or less than the molecular weight of the analyte (e.g., glucose) configured to be measured by device 20. The membrane is configured to restrict passage of (a) cells from outside device 20 into sampling region 30, and (b) cells 80 from within sampling region 30 to outside device 20.

Alternatively, cells 80 are disposed upon a scaffold, e.g., a silicone scaffold, and portions of cells 80 are encapsulated within respective biocompatible selectively-permeable membranes. In either embodiment, the membranes restrict passage of cells into sampling region 30 and also restrict passage of cells 80 from within sampling region 30 to outside device 20.

In an embodiment of the present invention, cells 80 are genetically engineered to express and secrete glucose oxidase (GOx) in-situ in region 30. This embodiment may be practiced in combination with techniques described in PCT Publication WO 06/006166 to Gross et al., and PCT Publication WO 07/110867 to Gross et al., and in the above-cited article by Scognamiglio et al.

In some embodiments, measuring glucose concentrations using FRET may be employed in combination with techniques described herein for measuring glucose using absorbance spectroscopy and/or polarimetry. Thus, combining techniques typically increases the effective signal-to-noise ratio of the device and its accuracy.

It is to be noted that the scope of the present invention includes the use of device 20 independently of cells 80, and that the proteins described herein may be disposed within sampling region 30. For example, during manufacture of device 20, genetically-engineered cells may produce the proteins described herein, which are then loaded into sampling region 30.

Alternatively or additionally, sampling region 30 comprises one or more types of microorganisms which respond to the specific analyte, e.g., glucose, in the blood of the subject, as described in U.S. Provisional Patent Application 60/588,211 to Gross et al.

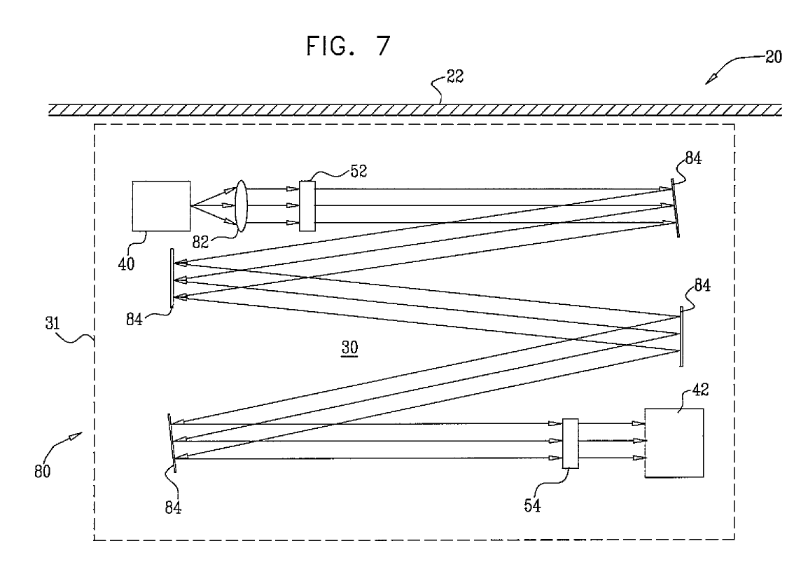

Reference is now made to FIG. 7, which is a schematic illustration of device 20 comprising a plurality of mirrors 84, in accordance with an embodiment of the present invention. Typically, mirrors 84 are configured to increase the length of the optical path of the light emitted by source 40. Light is reflected from mirrors 84, which extend the optical path of the light within region 30. Extending the optical path of the light thus satisfies the conditions (set forth in equation 1) for optimizing the illumination/detection of analyte concentrations in the region 30. Once the light is reflected from mirrors 84, it is passed though filter 54 and absorbed by detecting system 42, as described hereinabove.

Reference is now made to FIGS. 1-8. In the following techniques, device 20 comprises a filter (shown herein as filter 52) disposed adjacently to light source 40 (configuration not shown). Such a configuration of device 20 is applied in order to facilitate detection of glucose concentration by absorbance spectroscopy. It is to be noted that techniques described herein for measuring glucose concentration using absorbance spectroscopy comprise applying a range of wavelengths that are defined as being in the near infrared range (NIR), i.e., having wavelengths between 600 nm and 3000 nm. In some embodiments, light source 40 comprises a broadband LED, and filter 52 comprises a linearly tunable filter which disperses the light from the LED into narrow wavelength bands hi such an embodiment, detecting system 42 comprises a linear photodetector array, and each detector is positioned with respect to region 30 such that it detects a specific wavelength band.

FIG. 8 shows optical measuring device 20 comprising an annular, disc-shaped support 21 defining a disc-shaped sampling region 30, in accordance with an embodiment of the present invention. System 20 comprises a plurality of light sources 40 and a plurality of detecting systems 42, or sensors, which are disposed circumferentially along a wall 100 of support 21. Wall 100 surrounds sampling region 30. Support 21 facilitates a suitable spatial relationship (as shown) between sampling region 30, the plurality of light sources 40, and the plurality of detecting systems 42. In this manner, light sources 40 transmit light within sampling region 30 and each detecting system 42 receives at least a portion of the transmitted light that has passed through region 30.

As shown, a plurality of pairs of adjacently disposed light sources 40 and detecting systems 42 are disposed circumferentially along wall 100 by way of illustration and not limitation. For example, the plurality of light sources 40 may be disposed successively along a first portion of wall 100, and the plurality of detecting systems 42 may be disposed successively along a second portion of wall 100 that is opposite the first portion.

In some embodiments, one or more mirrors are disposed circumferentially along wall 100 of support 21. The one or more mirrors lengthen the optical path of the light emitted from the plurality of light sources (in a manner as described hereinabove with reference to FIGS. 3 and 7). Typically, the mirrors are disposed at a given geometrical orientation which optimizes the optical path length of the light transmitted from the plurality of light sources.

Support 21 has an upper surface 102 and a lower surface 104. An upper selectively-permeable membrane 110 is coupled to upper surface 102 and a lower selectively-permeable membrane 120 is coupled to lower surface 104. Typically, membranes 110 and 120 restrict passage of cells into sampling region 30. In some embodiments, membranes 110 and 120 comprise hydrophobic membranes, e.g., nitrocellulose membranes. Alternatively or additionally, membranes 110 and 120 comprise polyvinylidene difluoride, or PVDF, membranes. In some embodiments membranes 110 and 120 each have a molecular weight cutoff of around 500 kDa. It is to be noted, however, that embodiments described herein may be implemented independently of membranes 110 and 120.

Typically, interstitial fluid passively passes through membrane 110, through sampling region 30, and finally through membrane 120. Membranes 110 and 120 provide permeability for passage therethrough of certain constituents (e.g., small molecules such as glucose) of the interstitial fluid that have a molecular weight smaller than the molecular weight cutoff defined by membranes 110 and 120. For example, the molecular weight cutoff allows passage through membranes 110 and 120 of only glucose molecules present in the interstitial fluid and of other molecules having a molecular weight less than or generally equal to the molecular weight of the glucose molecule. That is, membranes 110 and 120 are configured to restrict passage therethrough into sampling region 30 of molecules or other body fluid components having a molecular or characteristic weight substantially greater than the molecular weight of a glucose molecule, e.g., cells.

Typically, the disc-shaped sampling region 30 has an tipper disc-shaped surface region (i.e., a first region exposed to the interstitial fluid) having a first surface area thereof, and a lower disc-shaped surface region (i.e., a second region exposed to the interstitial fluid) having a second surface area thereof. The first and second surface areas provide a combined large surface area for passive fluid transport through sampling region 30. In some embodiments, membrane 110 is disposed in the vicinity of the upper region of sampling region 30, and membrane 120 is disposed at the lower region of sampling region 30 (configuration shown). Sampling region 30 typically comprises optically-transparent and glucose-permeable material 70 (as described hereinabove with reference to FIGS. 1-4) independently of, or in combination with, membranes 110 and 120. In some embodiments, sampling region 30 comprises cells 80, as described hereinabove with reference to FIG. 6. In such an embodiment, membranes 110 and 120 restrict passage of cells 80 from within region 30 to outside device 20.

Support 21 has a height of between about 1 mm and 2 mm (typically, between about 1.5 mm and 2 mm) and a diameter of between about 4 mm and 12 mm (typically, between about 4 mm and 6 mm). As such, the upper and lower regions of sampling region 30 each have a diameter of between about 4 mm and 12 mm (typically, between about 4 mm and 6 mm). Typically, an average total surface area of support 21 is around 69 mm̂2, and an average combined surface area of the upper and lower regions of sampling region 30 is around 39 mm̂2.

Thus, the combined surface area provided for substance transport by the upper and lower regions of sampling region 30 is typically at least 50% (e.g., at least 70%) of a total surface area of optical measuring device 20. Typically, the combined surface area provided for substance transport by the upper and lower regions of sampling region 30 is typically less than 95% (e.g., less than 90%) of a total surface area of optical measuring device 20. Typically, both the upper and lower regions of sampling region 30 allow for passive fluid transport therethrough and into sampling region 30. In some embodiments, only one of the upper and lower regions of sampling region 30 allows for passive fluid transport therethrough and into sampling region 30.95, 90

It is to be noted that in some embodiments, FIGS. 1-3 show a lengthwise cross-section of the disc-shaped support 21 of FIG. 8, mutatis mutandis. Thus, FIGS. 1-3 may be interpreted as showing a flat, generally disc-shaped device, in which interstitial fluid flows through the top and/or bottom surfaces in each figure. Similarly, FIGS. 4-6, which show fluid flow through the left and right surfaces in each figure, may be generally disc-shaped and comprise large upper and lower surface areas for substance transport (as shown in FIG. 8).

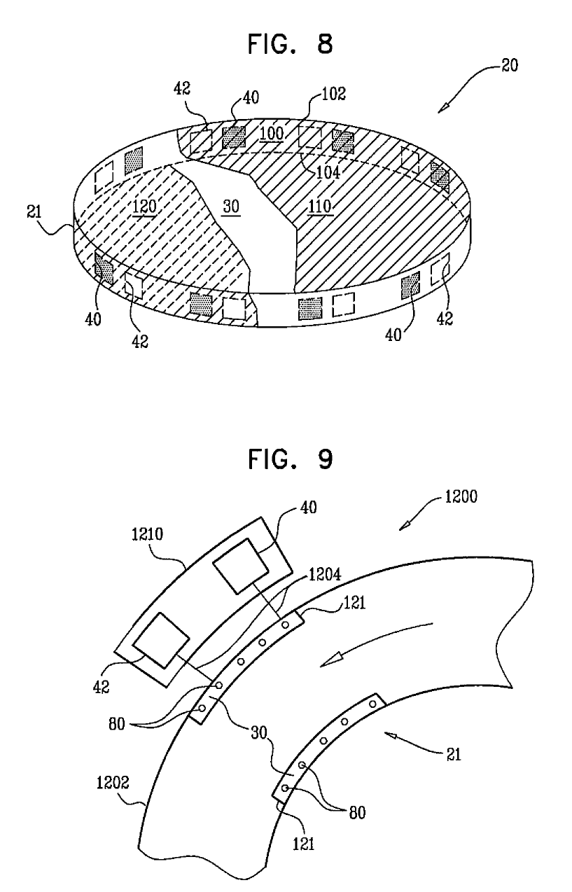

Reference is now made to FIG. 9, which is a cross-sectional schematic illustration of an optical measuring system 1200 comprising support 21 designated for implantation in a blood vessel 1202 of the subject, in accordance with an embodiment of the present invention. Typically, blood vessel 1202 includes a vena cava of the subject. Support 21 is shaped to define a cylindrical support 121 defining a cylindrical sampling region 30 which houses a plurality of genetically-engineered cells 80, as described hereinabove with reference to FIG. 6. In such an embodiment, sampling region 30 is disposed in a wall of cylindrical support 121. Typically, support 121 comprises a material which immunoisolates cells 80 from cells of the body of the subject. In some embodiments, support 121 is surrounded by a selectively-permeable membrane (not shown for clarity of illustration), which immunoisolates cells 80.

An electrooptical unit 1210 is disposed externally to blood vessel 1202 and houses light source 40 and detecting system 42. Unit 1210 is coupled to the support 121 via optical fibers 1204 which facilitate the propagation of light between unit 1210 and support 121.

Blood passes through vessel 1202 (in a direction as indicated by the arrow) and through a lumen defined by cylindrical support 121. As the blood passes through the lumen, components of blood are absorbed by support 121 and passes into sampling region 30. Cells 80 are engineered to produce a molecule (e.g., a protein) that is able to bind with an analyte in the blood and to undergo a conformational change in a detectable manner. In order to measure the conformational change of the proteins, and in turn, the amount of analyte in the blood, light is provided to sampling region 30 (in a manner as described hereinabove) by light source 40 via fibers 1204. Typically, detecting system 42 detects the conformational change, and in response, generates a signal indicative of a level of the analyte in the subject. Typically, but not necessarily, FRET techniques known in the art are used to detect the conformational change.

In some embodiments, cells 80 are genetically-engineered to secrete the protein into blood vessel 1202 and into the lumen defined by support 121. In such an embodiment, the lumen of support 121 functions as the sampling region. Light travels from unit 1210 toward the lumen of support 121 and is used to detect conformational changes of the secreted proteins within the lumen of vessel 1202.

Support 21 is shown as being cylindrical by way of illustration and not limitation. For example, support 21 may comprise a flexible disc-shaped housing comprising a gel that encapsulates the cells, and is disposed in vessel 1202 in a manner which reduces the incidence of clotting in vessel 1202 and reduces fibrosis of tissue around support 21.

In some embodiments, support comprises, by way of illustration and not limitation, agarose, silicone, polyethylene glycol, gelatin, an optical fiber capillary, a polymer, a co-polymer, and/or an alginate.

Reference is now made to FIGS. 1, 3, 4, 8, and 9. In some embodiments, device 20 and system 1200 lack filters 52 and 54, and techniques are applied in order to facilitate detection of glucose concentration by absorbance spectroscopy. In some embodiments, light source 40 comprises an array of narrow-band LEDs, and detecting system 42 comprises a photodetector. In some embodiments, light source 40 comprises a tunable laser diode, and detecting system 42 comprises a photodetector.

It is to be noted that region 30 may have any suitable length in accordance with a level of specificity and sensitivity of device 20, e.g., 10 mm. Increasing the length of region 30 increases the optical path length of the light, thereby increasing the sensitivity of device 20.

Reference is made to FIGS. 1-9. It is to be noted that for techniques by which glucose concentration is measured using absorbance spectroscopy, some scattering of light may occur in response to light deflecting from components disposed within the interstitial fluid. In some embodiments, the scattering induced by the absorbance spectroscopy comprises Raman scattering, which is observed when monochromatic light is incident upon optically-transparent (negligible absorption) media. In addition to the transmitted light, a portion of the light is scattered. Therefore, for some embodiments, device 20 comprises any suitable number of detectors which may be positioned at various locations with respect to device 20 in addition to detecting system 42, which is typically disposed in the optical path of the emitted beam. For example, detectors may be positioned in parallel and/or perpendicular orientations with the optical path (as defined by the arrows in each figure) of the emitted beam. Such a configuration of detectors enhances the signal to noise ratio of the measurement of glucose concentrations by device 20. In such an embodiment, light source 40 emits light in the near infrared (NIR) range, e.g., between 600 nm and 1000 nm.

Reference is now made to FIGS. 2, 6, and 7. A modulator may be added to filter 52 (adjacent to light source 40), to cause the polarization of the light to vary by a given angle. In some embodiments, the modulator comprises a Faraday rotator. In some embodiments, the modulator comprises a single Pockel's electro-optic effect modulator. In some embodiments, a closed-loop system using a Pockel's cell is used with a multiwavelength light source. In such an embodiment, the modulator may compensate for unwanted depolarization of the light within region 30. In some embodiments, the modulator comprises a liquid crystal based rotator in order to modulate the azimuth of the linearly polarized light emitted from source 40.

Reference is further made to FIGS. 1-9. In some embodiments, device 20 and system 1200 comprise a transmitter and a receiver. The transmitter is configured to be disposed in communication with detecting system 42, and the receiver is configured to be disposed remotely, e.g., outside the body of the subject. Typically, following the measurement of a parameter of the analyte in region 30, the transmitter transmits to the receiver an indication of the measured parameter. For embodiments in which the receiver is disposed outside the body of the subject, the receiver may notify the subject of the parameter in a humanly-perceptible manner. For example, the receiver may comprise a watch worn by the subject, and the watch may be configured to display the measured parameter on a display.

It is to be noted that the scope of the present invention includes the use of any of the optical sensing devices (independently or in combination) described with respect to FIGS. 1-9 for sensing constituents of fluids other than glucose. For example, apparatus described herein may be used to detect levels of calcium ions present in the fluid of the subject, mutatis mutandis.

It is to be further noted that the scope of the present invention includes the use of any of the optical sensing devices (independently or in combination) described with respect to FIGS. 1-9 for measuring the concentration of a particular analyte in any fluid of the body of the subject.

The scope of the present invention includes embodiments described in one or more of the following:
U.S. Provisional Patent Application 60/588,211 to Gross et al., entitled, “Implantable sensor,” filed Jul. 14, 2004;
U.S. Provisional Patent Application 60/658,716 to Gross et al., entitled, “Implantable fuel cell,” filed Mar. 3, 2005;
PCT Patent Application PCT/IL2005/000743 to Gross et al., entitled “Implantable power sources and sensors,” filed Jul. 13, 2005;
U.S. Provisional Patent Application 60/786,532 to Gross et al., entitled, “Implantable sensor,” filed Mar. 28, 2006;
PCT Patent Application PCT/IL2007/000399 to Gross, entitled “Implantable sensor,” filed Mar. 28, 2007.

All of these applications are incorporated herein by reference.

For some applications, techniques described herein are practiced in combination with techniques described in one or more of the references cited in the Background section and the Cross-references section of the present patent application. All references cited herein, including patents, patent applications, and articles, are incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
a support configured to be implanted within a body of a subject;
a sampling region coupled to the support, the apparatus configured to passively allow passage through the sampling region of at least a portion of fluid from the subject; and
an optical measuring device in optical communication with the sampling region, comprising:
at least one light source configured to transmit light through at least a portion of the fluid, and
at least one sensor configured to measure a parameter of the fluid by detecting light passing through the fluid.

2. The apparatus according to claim 1, wherein the portion of the fluid includes glucose, and wherein the apparatus is configured to passively allow passage of the glucose through the sampling region.

3. The apparatus according to claim 1, wherein the parameter of the fluid includes glucose concentration, and wherein the optical measuring device is configured to measure a concentration of glucose in the fluid.

4. The apparatus according to claim 1, wherein the apparatus is configured for subcutaneous implantation within the subject.

5. The apparatus according to claim 1, wherein the fluid includes components of interstitial fluid of the subject, and wherein the apparatus is configured to facilitate a measurement of a parameter of the interstitial fluid of the subject.

6. The apparatus according to claim 1, wherein the light source comprises one or more light sources selected from the group consisting of: a light emitting diode (LED), an organic light emitting diode (OLED), a laser diode, and a solid-state laser.

7. The apparatus according to claim 1, wherein the light source is configured to emit visible light.

8. The apparatus according to claim 1, wherein the light source is configured to emit infrared light.

9. The apparatus according to claim 1, further comprising a drug administration unit configured to administer a drug in response to the measured parameter.

10. The apparatus according to claim 1, wherein the optical measuring device comprises an absorbance spectrometer.

11. The apparatus according to claim 1, further comprising a housing coupled to the support and surrounding the sampling region, the housing having at least one opening formed therein configured for passage of the fluid therethrough and into the housing.

12. The apparatus according to claim 1, further comprising a transmitter and a receiver, the transmitter configured to be in communication with the sensor, and the receiver configured to be disposed at a site outside the body of the subject, wherein the transmitter is configured to transmit the measured parameter to the receiver.

13. The apparatus according to claim 1, wherein:
the support is shaped to define a cylindrical support defining a lumen thereof, and
the sampling region is disposed within the lumen.

14. The apparatus according to claim 1, further comprising cells disposed within the sampling region, the cells being genetically engineered to produce, in situ, a protein configured to facilitate a measurement of the parameter of the fluid.

15. The apparatus according to claim 1, wherein the light source comprises a plurality of light sources, and wherein the sensor comprises a plurality of photodetectors.

16. The apparatus according to claim 1, wherein the light source is configured to emit polarized light, and wherein the apparatus further comprises at least one first polarizing filter having an orientation thereof and configured to filter the polarized light emitted from the light source into the sampling region.

17. The apparatus according to claim 1, wherein:
the support is shaped to define a wall thereof surrounding the sampling region,
the at least one light source comprises a plurality of light sources disposed along the wall of the support and configured to transmit light through the sampling region, and
the at least one sensor comprises a plurality of sensors disposed along the wall of the support and configured to receive at least a portion of the light passing through the fluid.

18. The apparatus according to claim 1, wherein the sampling region comprises a permeable material selected from the group consisting of: agarose, silicone, polyethylene glycol, gelatin, an optical fiber capillary, a polymer, a co-polymer, an extracellular matrix, and alginate, the permeable material being positioned to passively allow passage therethrough of the portion of fluid in the sampling region.

19. The apparatus according to claim 18, wherein the material comprises an optically-transparent and glucose-permeable material.

20. The apparatus according to claim 18, wherein the material is configured to restrict passage of cells into and out of the sampling region.

21. The apparatus according to claim 1, further comprising at least one selectively-permeable membrane coupled to the support.

22. The apparatus according to claim 21, wherein the membrane is configured to restrict passage of cells into and out of the sampling region.

23. The apparatus according to claim 21, wherein the support has a first surface and a second surface, and wherein the at least one selectively-permeable membrane comprises:
a first selectively-permeable membrane coupled to the first surface; and
a second selectively permeable membrane coupled to the second surface.

24. The apparatus according to claim 1, wherein:
the fluid includes components of blood of the subject,
the support is configured for implantation within a blood vessel of the subject, and
the apparatus is configured to facilitate a measurement of a parameter of blood of the subject.

25. The apparatus according to claim 24, wherein the blood vessel includes a vena cava of the subject, and wherein the support is configured for implantation within the vena cava of the subject.

26. The apparatus according to claim 24, wherein the optical measuring device is configured to be disposed externally to the blood vessel, and wherein the optical measuring device is configured to be in optical communication with a vicinity of the blood vessel in which the support is implanted.

27. The apparatus according to claim 24, wherein the support is shaped to define a cylindrical support, the cylindrical support defining a lumen thereof that surrounds the sampling region.

28. The apparatus according to claim 24, further comprising at least one optical fiber, wherein the optical fiber is coupled at a first end to the optical measuring device, and at a second end to the support, and wherein light from the light source is provided to the sampling region via the optical fiber.

29. The apparatus according to claim 24, wherein the parameter of the blood includes a level of glucose in the blood, and wherein the apparatus is configured to facilitate a measurement of the level of glucose in the blood of the subject.

30. The apparatus according to claim 1, wherein the apparatus further comprises a tunable filter configured to refract the light emitted from the light source into a plurality of monochromatic bands.

31. The apparatus according to claim 30, wherein the tunable filter comprises a Faraday rotator.

32. The apparatus according to claim 30, wherein the sensor comprises a plurality of photodetectors, each photodetector detecting a respective one of the plurality of monochromatic bands.

33. The apparatus according to claim 1, further comprising at least one reflector, configured to reflect to the sensor light emitted from the light source that has passed through the sampling region.

34. The apparatus according to claim 33, wherein the at least one reflector comprises a plurality of reflectors, wherein each one of the plurality of reflectors is disposed at a respective location with respect to the sampling region, and wherein the plurality of reflectors lengthens an optical path between the light source and the sensor.

35-98. (canceled)
